SEQUENCE LISTING (2 sequences)

SEQ ID NO: 1 - protein, 8 aa, Homo sapiens
VRQTWKLE

SEQ ID NO: 2 - protein, 8 aa, Homo sapiens
MRLGPRWK

(19) United States
(12) Patent Application Publication
(10) Pub. No.: US 2014/0302526 A1
(43) Pub. Date: Oct. 9, 2014

(54) ZAP-70 DETECTION IN CHRONIC LYMPHOCYTIC LEUKEMIA

(71) Applicant: Creatv Micro Tech, Inc., Potomac, MD (US)

(72) Inventors: Cha-Mei Tang, Potomac, MD (US); Peixuan Zhu, Derwood, MD (US)

(21) Appl. No.: 14/355,247

(22) PCT Filed: Nov. 2, 2012
(86) PCT No.: PCT/US2012/063383

§ 371 Date: Apr. 30, 2014

Related U.S. Application Data
(60) Provisional application No. 61/555,199, filed on Nov. 3, 2011.

(51) Int. Cl. G01N 33/574

(52) U.S. Cl. CPC G01N 33/57407
USPC 435/7.4

(57) ABSTRACT

Detection of ZAP-70 expression provides important information about disease progression and overall survival in patients with chronic lymphocytic leukemia (CLL). The invention provides methods for diagnosing CLL in a subject, as well as methods for clearly distinguishing CLL patients with aggressive form of the disease. A consistent number of B cells from patient blood is isolated and lysed to release all of the intracellular ZAP-70 protein. The released ZAP-70 protein is subsequently extracted by immunomagnetic separation followed by detection with fluorescence immunosandwich assay. The ZAP-70 fluorescence signal is measured with Signalyte™-II spectrofluorometer. The VeriZAP™ assay is a simple, reliable, and reproducible method for quantitative detection of ZAP-70 in patient leukemic cells, and can be used as a prognostic test to distinguish indolent versus aggressive CLL patients.

### CROSS-REFERENCE TO RELATED APPLICATION

This application claims benefit under 35 U.S.C. §119(e) of U.S. provisional application Ser. No. 61/555,199, filed Nov. 3, 2011, which is incorporated herein by reference in its entirety.

### FIELD OF THE INVENTION

The present invention provides methods for diagnosing disease in a subject based by determining the amounts of one or more selected proteins in a test sample. In one aspect, the methods of the present invention can be used to discern between different forms of the same disease based on the relative amounts of the one or more selected proteins in the test sample. For example, aggressive disease can be distinguished from an indolent form of the disease. The diagnostic methods of the present invention are based on a novel immunoassay system that permits the quantitative detection of a selected protein in a test sample, and materials and equipment used therein. In one aspect of the invention, a fluorescence immunoassay, reagents and fluorometer are provided for the detection of ZAP-70 protein in a test sample, such as a population of B cells. The immunoassay can be used, for example, to clearly distinguish patients having the aggressive form of chronic lymphocytic leukemia from those with the non-aggressive form of the disease. The immunoassays of the invention can also be applied to the detection of other protein biomarkers from cells, tissues and body fluids, such as tumor cells, fetal cells, epithelial cells, blood cells, and the like. Body fluids can be blood, plasma, serum, spinal fluid, saliva, urine, tears, mucus, etc.

### DESCRIPTION OF RELATED ART

Chronic lymphocytic leukemia (CLL) is heterogeneous disease with high individual variability in clinical course. Some patients survive for decades without needing treatment, whereas others progress rapidly in the disease and require early aggressive therapies. In the past ten years, a number of prognostic markers have been reported for prediction of outcome and response in early-stage CLL. These prognostic markers fall into the following two main categories, genetic markers and protein surrogate markers. The genetic markers include somatic mutational status of the immunoglobulin variable heavy chain region gene (IgVH) (Hamblin et al., 1999, Blood 94:1848-1854), genomic aberrations such as deletions of 11q and 17p (Döhner et al., 2000, N Engl J Med 343:1910-1916; Krober et al., 2002, Blood 100:1410-1416), loss or mutation of the p53 gene, microRNA expression profiles (Calin et al., 2004, Proc Natl Acad Sci USA 101:11755-11760; Moussay et al., 2011, Proc Natl Acad Sci USA 108:6573-6578), and C-334 methylation status (Corcoran et al., 2005, Haematologica 90:1078-1088). The most established genetic predictor of disease progression is presence or absence of somatic mutations in the IgVH region of CLL leukemic cells. Absence of mutations in the leukemic cells (IgVH-unmutated CLL) often correlates with aggressive disease, whereas the presence of mutations in the leukemic cells (IgVH-mutated CLL) correlates with longer survival in 50-70% CLL patients. However, because IgVH mutation analysis is not routinely available in many clinical laboratories, several protein surrogate markers for prediction of CLL have been reported, such as CD38 antigen expression (Damle et al., 1999, Blood 94:1840-1847; Diirig et al., 2002, Leukemia 16:30-35), Zeta-chain-associated protein 70 (ZAP-70) (Crespo et al., 2003, N Engl J Med 348:1764-1775; Wiestner et al., 2003, Blood 101:4944-4951) and lipoprotein lipase (LPL) (Van Bockstaele et al., 2007, Clin Chem. 53:204-212).

Overexpression of the intracellular protein ZAP-70 was identified in B cells in a subset of patients with chronic lymphocytic leukemia (CLL) by Louis M. Staudt et al. (U.S. Pat. No. 7,329,502, issued on Feb. 12, 2008, ZAP-70 Expression as a Marker for Chronic Lymphocytic Leukemia/Small Lymphocytic Lymphoma (CLL/SLL)). To date, it has been accepted that the most effective surrogate marker of CLL outcome and response is expression of ZAP-70. A pilot gene expression profiling study of 28 patients has shown that ZAP-70 was more highly expressed in Ig-unmutated CLL than in Ig-mutated CLL (Rosenwald et al., 2001, J Exp Med. 194:1639-1647). A follow-up study with 107 patients showed that ZAP-70 was expressed 5.5-fold higher in Ig-unmutated CLL than in Ig-mutated CLL (Wiestner et al., 2003, Blood 101:4944-4951). Comparative analysis revealed a high degree of correlation between ZAP-70 expression and IgVH mutation status in 93% of patients. Patients with 20% leukemic cells expressing ZAP-70 were unmutated in IgVH; in contrast, patients with less than 20% leukemic cells expressing ZAP-70 were mutated in IgVH (Crespo et al., 2003, N Engl J Med 348:1764-1775; Wiestner et al., 2003, Blood 101:4944-4951). Further study showed that ZAP-70 was expressed in 117 of the 164 patients with an unmutated IgVH gene (71%), but in only 24 of the 143 patients with a mutated IgVH gene (17%) (Rassenti, et al., 2004, N Engl J Med 351:893-901). Thus, determination of ZAP-70 expression level would yield important prognostic information for patients with CLL.

Despite the fact that the prognostic value of ZAP-70 for CLL is widely accepted, researchers have criticized the current methods for detection of ZAP-70. Many methods have been described for detection of ZAP-70 expression, including microarray-based gene expression profiling (Klein et al., 2001, J Exp Med 194:1625-1638; Rosenwald et al., 2001, J Exp Med 194:1639-1647); flow cytometry (U.S. Pat. No. 7,759,076; Crespo et al., 2003, N Engl J Med 348:1764-1775; Wiestner et al., 2003, Blood 101:4944-4951), ELISA (Pryshchep et al., 2010, Circ Res 106:769-778), reverse transcriptase-PCR (Stamatopoulos et al., 2007, Clin Chem 53:1757-1766), immunohistochemistry (Carreras et al., 2005, J Pathol 205:507-513; Admirand et al., 2010, Mod Pathol 23:1518-1523) and Western blot analysis (Chen et al., 2002, Blood 100:4609-4614). Flow cytometry is commonly used for measurement of ZAP-70 in CLL cells because it offers the advantage of allowing specific gating on different cell populations of interest. Several fluorescence-conjugated anti-ZAP-70 antibodies are commercially available for testing ZAP-70 in flow cytometry, such as 2F3.2-FITC (Upstate Biotechnology, Waltham, Mass.), 1E7.2-FITC (e-Bioscience, San Diego, Calif.), and 1E7.2-Alexa Fluor 488 (Caltag, Burlingame, Calif.). A comparative study using flow cytometry showed that 1E7.2-Alexa Fluor 488 had the highest binding affinity of the tested anti-ZAP-70 antibodies (Preobrazhensky & Bahler, 2008, Cytometry B Clin Cytom 74:118-127). However, attempts to implement a ZAP-70 approach in clinical flow cytometry laboratories have been problematic since many commercially available antibodies give unreliable results (Admirand et al., 2010, Mod Pathol. 23:1518-1523). Due to these variations, it is difficult to standardize the protocols of flow cytometry for detection of ZAP-70. To date, this test has not undergone national standardization. Further, flow cytometry also suffers from lack of establishment of a clear cutoff value. The flow cytometry assay is based on detection of the number of B cells with detectable ZAP-70 marker rather than detection of the ZAP-70 protein itself. The distribution of ZAP-70-positive cells (usually reported as percentage) appears as a continuous curve in the population of CLL patients (Rassenti et al., 2004, NEJM 351: 892-901). Therefore, a clear cutoff cannot be established for distinguishing the aggressive type from the indolent type of CLL (U.S. Pat. No. 7,759,076).

In addition to flow cytometry, several alternative assay formats have been available for the detection of human ZAP-70, such as ELISA, bead-sandwich, cytometric bead array and fluorescence polarization. These methods are designed for qualification of total human ZAP-70 or for assaying kinase activity, and they are typically expensive and require assay times of from 4 hours to overnight. Translating these methods into the clinic laboratory as routine diagnostic tools has not been achieved because they have not been standardized and validated, resulting in the lack reproducibility and multiple issues related to assay time, cost, complexity and data analysis. Currently, a reliable and quantitative method for assessing ZAP-70 in leukemic cells is lacking. Thus, development of a new method for quantitative detection of ZAP-70 could make this marker widely available for diagnosis and prognosis of CLL in the clinical setting.

### SUMMARY OF THE INVENTION

The present invention generally provides methods for diagnosing disease in a subject based by determining the amounts of one or more selected proteins in a test sample. In one aspect, the methods of the present invention can be used to discern between different forms of the same disease based on the relative amounts of the one or more selected proteins in the test sample. For example, aggressive disease can be distinguished from an indolent form of the same disease. The present invention further generally provides novel immunoassays that can be used in the diagnostic methods of the present invention. These immunoassays can be used to detect one or more selected proteins in a test sample.

In a first embodiment, the present invention is directed to methods for diagnosing chronic lymphocytic leukemia (CLL) in a subject. In one aspect, the method comprises preparing a ZAP-70 ratio M/N, wherein the ratio is prepared by quantitatively determining the amount of ZAP-70 M in a lysate of a test sample from a test subject, and dividing the determined amount M by a control amount N, wherein the control amount N is obtained by quantitatively determining the amount of ZAP-70 in a lysate of a control sample of the same identity as the test sample from a control subject without CLL, wherein when the ZAP-70 ratio M/N is greater than about 2.0, the test subject is diagnosed as having an aggressive form of CLL, and wherein when the ZAP-70 ratio M/N is less than about 2.0 the test subject is diagnosed as having an indolent form of CLL. In a preferred example of this aspect, the test sample and control sample are a predetermined number of B cells. In another preferred example of this aspect, the control amount N is obtained by quantitatively determining the amount of ZAP-70 in a lysate of a control sample of the same identity as the test sample from a pool of control subjects without CLL.

In another aspect of the diagnostic method of the first embodiment, the method comprises (a) preparing a normalized ZAP-70 value X, wherein the normalized value is prepared by (i) quantitatively determining the amount of ZAP-70 M in a lysate of a test sample from a test subject, (ii) quantitatively determining the amount of a control marker P in the same lysate, and (iii) dividing the amount determined for ZAP-70 by the amount determined for the control marker to obtain the normalized ZAP-70 value where X=M/P, (b) preparing a reference ZAP-70 value Y, wherein said reference value is prepared by (i) quantitatively determining the amount of ZAP-70 N in a lysate of a control sample of the same identity as the test sample from a control subject without CLL, (ii) quantitatively determining the amount of the control marker R in the same lysate, and (iii) dividing the amount determined for ZAP-70 by the amount determined for the control marker to obtain the reference ZAP-70 value where Y=N/R, and (c) obtaining a ZAP-70 ratio X/Y by dividing the normalized ZAP-70 value X by the reference ZAP-70 value Y, wherein when the ZAP-70 ratio X/Y is greater than about 2.0, the test subject is diagnosed as having an aggressive form of CLL, and wherein when the ZAP-70 ratio X/Y is less than about 2.0 the test subject is diagnosed as having an indolent form of CLL. In a preferred example of this aspect, the test sample and the control sample are a predetermined number of B cells. In another preferred example of this aspect, the lysate of a control sample of the same identity as the test sample is from a pool of control subjects without CLL.

In a second embodiment, the present invention is directed to immunoassays that can be used in the detection of selected proteins in a test sample. The immunoassays can be used in separately or in conjunction with the diagnostic methods of the present invention. In one aspect of this embodiment, the immunoassays can be used for the detection of ZAP-70 in a test sample, such as a population of B cells. The B cells may be from patients with chronic lymphocytic leukemia (CLL), for example (e.g., leukemic B cells). In conjunction with the detection of ZAP-70 in a test sample, the immunoassay is termed the VeriZAP™ assay system. The novel immunoassay system has the advantage of not including a step of conventional flow cytometry. The immunoassay system comprises a method for detection of ZAP-70 protein in a liquid-phase from a lysed population of cells (e.g., leukemic B cells) rather than counting whole cells expressing the detectable ZAP-70 marker.

Another aspect of the invention includes reagents, protocols and instrumentation useful in performing the immunoassays, including preparation of populations of B cells from a blood sample, extraction of intracellular ZAP-70 from the B cells, a liquid-phase fluorescence antibody assay, and measurement of ZAP-70 signal. In particular, populations of cells (e.g., leukemic B cells) that can be subjected to the immunoassays of the invention can be isolated from patient blood by a variety of methods. The purified cells are lysed to release all of the intracellular ZAP-70 proteins into a cell lysate. The released ZAP-70 proteins are extracted by immunomagnetic separation followed by detection with the fluorescence immunoassay. The invention provides a rapid and sensitive assay system for quantitative detection of ZAP-70 expression in the cells without use of the conventional flow cytometry. Further, the immunoassay system is simple, reliable and reproducible with the added advantage of eliminating multiple steps in sample pre-treatment, such as red blood cell lysis, cell fixation or permeabilization. The fluorescent ZAP-70 signal achieved using the immunoassay system of the invention is linear over a wide dynamic range, which enables quantitative assessment of small changes in ZAP-70 expression over the course of the disease and in response to therapeutic intervention.

A first specific example of the diagnostic method of the present invention is directed to a method of diagnosing chronic lymphocytic leukemia (CLL) in a subject, comprising preparing a ZAP-70 ratio M/N, wherein the ratio is prepared by quantitatively determining the amount of ZAP-70 M in a lysate of a predetermined number of B cells from a test subject, and dividing the determined amount M by a control amount N, wherein the control amount N is obtained by quantitatively determining the amount of ZAP-70 in a lysate of B cells of the same predetermined number from a control subject without CLL, wherein when the ZAP-70 ratio M/N is greater than about 2.0, the test subject is diagnosed as having an aggressive form of CLL, and wherein when the ZAP-70 ratio M/N is less than about 2.0 the test subject is diagnosed as having an indolent form of CLL.

In aspects of this example, the control amount N is obtained by quantitatively determining the amount of ZAP-70 in a lysate of B cells of the same predetermined number from a pool of control subjects without CLL.

In aspects of this example, the amount of ZAP-70 in a lysate is determined by:

a) collecting a predetermined number of B cells,

b) lysing the B cells,

c) capturing ZAP-70 from the cell lysate of b), and

d) measuring the amount of ZAP-70 captured in c).

In aspects of this example, B cells are collected using a technique selected from the group consisting of immunomagnetic separation, Ficoll gradient followed by immunomagnetic separation, depletion of non-B cells (negative selection), and fluorescence-activated cell sorting (FACS).

In aspects of this example, ZAP-70 is captured using one or more members selected from the group consisting of capture antibody-coated magnetic beads, aptamer-coated magnetic beads, ligand-coated magnetic beads, capture antibody-coated glass beads, aptamer-coated glass beads, ligand-coated glass beads, a capture antibody-containing ferrofluid, an aptamer-containing ferrofluid, a ligand-containing ferrofluid, a capture antibody-coated microfluidic chip, an aptamer-coated microfluidic chip and a ligand-coated microfluidic chip.

In aspects of this example, the amount of ZAP-70 is measured by fluorescent sandwich assay or electrochemiluminescence. The fluorescent sandwich assay uses a detector antibody labeled with a detectable label selected from the group consisting of a fluorescent dye, a dye particle and quantum dots. A Signalyte®-II spectrofluorometer detection instrument, fluorescent plate reader or microfluidic chip is used to measure the amount of detectable label in the fluorescent sandwich assay.

In aspects of this example, ZAP-70 is captured using capture antibody-coated magnetic beads, the capture antibody is selected from the group consisting of antibody 2F3.2, antibody 1E7.2, and SBZAP, and the detector antibody is selected from the group consisting of antibody 2F3.2, antibody 1E7.2, and SBZAP. Further, the fluorescence dye is selected from the group consisting of Texas Red, allophycocyanin (APC), Cy™-5, PE-Cy5.5, Dylight 649, DyLight 638/658, DyLight 654/673, DyLight 692/712, Alexa Fluor 590/617, Alexa Fluor 612/626, Alexa Fluor 632/647, Alexa Fluor 633/647, Alexa Fluor 650/665, and Alexa Fluor 663/690.

A second specific example of the diagnostic method of the present invention is directed to a method of diagnosing chronic lymphocytic leukemia (CLL) in a subject, comprising

(a) preparing a normalized ZAP-70 value X, wherein the normalized value is prepared by (i) quantitatively determining the amount of ZAP-70 M in a lysate of a predetermined number of B cells from a test subject, (ii) quantitatively determining the amount of a control marker P in the same lysate, and (iii) dividing the amount determined for ZAP-70 by the amount determined for the control marker to obtain the normalized ZAP-70 value where X=M/P,

(b) preparing a reference ZAP-70 value Y, wherein said reference value is prepared by (i) quantitatively determining the amount of ZAP-70 N in a lysate of B cells of the same predetermined number from a control subject without CLL, (ii) quantitatively determining the amount of the control marker R in the same lysate, and (iii) dividing the amount determined for ZAP-70 by the amount determined for the control marker to obtain the reference ZAP-70 value where Y=N/R, and

(c) obtaining a ZAP-70 ratio X/Y by dividing the normalized ZAP-70 value X by the reference ZAP-70 value Y,

wherein when the ZAP-70 ratio X/Y is greater than about 2.0, the test subject is diagnosed as having an aggressive form of CLL, and wherein when the ZAP-70 ratio X/Y is less than about 2.0 the test subject is diagnosed as having an indolent form of CLL.

In aspects of this example, the lysate of B cells of the same predetermined number is from a pool of control subjects without CLL.

In aspects of this example, the control marker is a protein selected from the group consisting of a tyrosine kinase protein, a human leukocyte differentiation antigen, and a transmembrane receptor proteins.

In aspects of this example, the amounts of ZAP-70 and control marker in a lysate of a predetermined number of B cells are determined by:

a) collecting a predetermined number of B cells,

b) lysing the B cells,

c) capturing ZAP-70 and the control marker from the cell lysate of b), and

d) measuring the amount of ZAP-70 and the control marker captured in c).

In aspects of this example, the B cells are collected using a technique selected from the group consisting of immunomagnetic separation, Ficoll gradient followed by immunomagnetic separation, depletion of non-B cells (negative selection), and fluorescence-activated cell sorting (FACS).

In aspects of this example, ZAP-70 and the control marker are independently captured using one or more members selected from the group consisting of capture antibody-coated magnetic beads, aptamer-coated magnetic beads, ligand-coated magnetic beads, capture antibody-coated glass beads, aptamer-coated glass beads, ligand-coated glass beads, a capture antibody-containing ferrofluid, an aptamer-containing ferrofluid, a ligand-containing ferrofluid, a capture antibody-coated microfluidic chip, an aptamer-coated microfluidic chip and a ligand-coated microfluidic chip.

In aspects of this example, the amount of ZAP-70 and the control marker are independently measured by fluorescent sandwich assay or electrochemiluminescence. The fluorescent sandwich assay uses a detector antibody labeled with a fluorescent material selected from the group consisting of a fluorescent dye, a dye particle and quantum dots. A Signalyte®-II spectrofluorometer detection instrument, fluorescent plate reader or microfluidic chip is used to measure the amount of fluorescence in the fluorescent sandwich assay.

In aspects of this example, ZAP-70 is captured using capture antibody-coated magnetic beads and the capture antibody is selected from the group consisting of antibody 2F3.2, antibody 1E7.2, and SBZAP. The detector antibody is selected from the group consisting of antibody 2F3.2, antibody 1E7.2, and SBZAP. Further, the fluorescence dye is selected from the group consisting of Texas Red, allophycocyanin (APC), Cy™-5, PE-Cy5.5, Dylight 649, DyLight 638/658, DyLight 654/673, DyLight 692/712, Alexa Fluor 590/617, Alexa Fluor 612/626, Alexa Fluor 632/647, Alexa Fluor 633/647, Alexa Fluor 650/665, and Alexa Fluor 663/690.

A third specific example of the diagnostic method of the present invention is directed to a method of diagnosing chronic lymphocytic leukemia (CLL) in a subject, comprising

a) preparing a ZAP-70 ratio M/N, wherein the ratio is prepared by quantitatively determining the amount of ZAP-70 M in a lysate of a predetermined number of B cells from a test subject by the following steps:
i) collecting a predetermined number of B cells from the test subject,
ii) lysing the B cells of i),
iii) capturing ZAP-70 from the B cell lysate of ii) using magnetic beads coated with a capture antibody, wherein the capture antibody has binding specificity for ZAP-70,
iv) forming an immunosandwich complex by adding a detector antibody to iii), wherein the detector antibody has binding specificity for ZAP-70 and wherein the detector antibody is labeled with a fluorescent dye,
v) collecting the magnetic beads from iv), and
vi) detecting and measuring fluorescent signal in v), wherein the amount of fluorescent signal corresponds to the amount of ZAP-70 M,

b) dividing the determined amount M by a control amount N, wherein the control amount N is obtained by quantitatively determining the amount of ZAP-70 in a lysate of B cells of the same predetermined number from a control subject without CLL by the following steps:
i) collecting B cells of the same predetermined number from a control subject or pool of control subjects,
ii) lysing the B cells of i),
iii) capturing ZAP-70 from the B cell lysate of ii) using magnetic beads coated with a capture antibody, wherein the capture antibody has binding specificity for ZAP-70,
iv) forming an immunosandwich complex by adding a detector antibody to iii), wherein the detector antibody has binding specificity for ZAP-70 and wherein the detector antibody is labeled with a fluorescent dye,
v) collecting the magnetic beads from iv), and
vi) detecting and measuring the amount of fluorescent signal in v), wherein the amount of fluorescent signal corresponds to the control amount N,

wherein when the ZAP-70 ratio M/N is greater than about 2.0, the test subject is diagnosed as having an aggressive form of CLL, and wherein when the ZAP-70 ratio M/N is less than about 2.0 the test subject is diagnosed as having an indolent form of CLL.

In aspects of this example, the capture antibody is independently selected from the group consisting of antibody 2F3.2, antibody 1E7.2, and SBZAP. The detector antibody is independently selected from the group consisting of antibody 2F3.2, antibody 1E7.2, and SBZAP.

### BRIEF DESCRIPTION OF THE DRAWINGS

FIG. 1. TCR signaling cascade and ZAP-70 function. ZAP-70 is a member of the protein tyrosine kinase family, which is normally expressed in T cells and natural killer cells. A high level of ZAP-70 expression appears restricted to T-cell proliferative diseases and a subgroup of CLL. Activation of ZAP-70 involves MHC Class II 1001 and peptide 1002, CD3 1005, and TCR 1003, CD4 1006 and Lck 1007. ZAP-70 1008 is phosphorylated on tyrosine residues upon TCR stimulation and binds CD3-zeta 1004, and then interact with adaptor proteins 1009, and send messages to the nucleus 1010, where the transcription of several genes 1011, which allow the T cells to differentiate, proliferate and secrete a number of cytokines, is activated.

FIG. 2. Schematic diagram of three isoforms of ZAP-70 and location of antibody epitopes. The numbers under the protein indicate amino acid positions. Isoform-1 2007 is a full length ZAP-70 protein (619 amino acids), while Isoform-2 2008 (312 amino acids) and Isoform-3 2009 (493 amino acids) are truncated proteins. Differences in amino acid sequences are also observed between the isoform-1 (positions 127-134; SEQ ID NO:1) and the isoform-3 (positions 1-8; SEQ ID NO:2). Clone 2F3.2 antibody was generated against GST-fusion protein 2001 corresponding to amino acids 1-254 of Isoform-1. Clone 1E7.2 antibody was generated against a KLH-peptide sequence 2003 corresponding to the human ZAP-70 Isoform-1 amino acids 282-307. Clone SBZAP was generated was generated against a KLH-peptide sequence 2002 corresponding to human ZAP-70 Isoform-1 amino acids 280-309.

FIG. 3. Schematic diagram illustrating the principle of detecting ZAP-70 in the cell lysate. An anti-ZAP-70 capture antibody is first immobilized on the magnetic beads 3001. The ZAP-70 protein 3002 in cell lysate is specifically captured by the capture antibody-bound magnetic beads. Other unbound proteins and contaminates 3003 are washed away. A fluorescent detector antibody 3004 recognizes the bead-captured ZAP-70, forming an immunosandwich complex 3005. Unbound detector antibodies are washed away. The detector antibody is further eluted from the magnetic beads. Distinct fluorescence signal 3006 of the detector antibody, corresponding to the ZAP-70 concentration, is measured using a quantitative and sensitive fluorescence detection instrument, for example a Signalyte™-II spectrofluorometer. By comparison of fluorescence intensities from sample and standard material, the results are reported in quantitative format.

FIG. 4. Selection of antibody for the detection of ZAP-70. Clones 2F3.2 and SBZAP were used as capture antibodies, whereas clone 1E7.2 was used as a detector antibody. Two ZAP-70-positive samples (Jurkat cell lysate, P8 CLL cell lysate) and a PBS buffer control were tested. The clone 2F3.2 antibody produced higher detection signal than the clone SBZAP antibody for both Jurkat cell lysate (1.6-fold) and P8 cell lysate (4.3-fold). The PBS control showed low background signal, indicating that the assay was highly specific for detection of ZAP-70.

FIG. 5. Selection of fluorescence dyes for the detector antibody. Clone 1E7.2 antibody was used as a detector antibody. This detector antibody was conjugated with four different fluorescence dyes, PE-Cy5.5, allophycocyanin (APC), Cy5 and Dylight 649, respectively. Jurkat cell lysate was used as positive control to evaluate the detection sensitivity, whereas PBS solution was used as negative control to evaluate the non-specific background noise. The Y-axis indicates the signal-to-noise ratio (S/N). Although Cy5-conjugated antibody produced the highest signal, it also produced higher background noise, resulting lower S/N ratio. The PE-Cy5.5- and Dylight 649-conjugated antibodies produced lower background noise, resulting in better S/N ratios.

FIGS. 6A&B. Detection of ZAP-70 in Jurkat cell lysate. The plots show the relationships of normalized fluorescence signal (NFI) and the number of Jurkat cells used for preparation of cell lysate samples. (A) Low cell concentration in a range of 0-1,000 cells/reaction. (B) High cell concentration in range of 0-40,000 cells/reaction. To precisely measure each sample and maximize detection sensitivity without saturation, two different measurement times were used for the lower concentration range (3,000 ms) and high concentration range (10 ms).

FIGS. 7A&B. Detection of recombinant ZAP-70 protein. The plots show the relationships of NFI and input concentrations of recombinant ZAP-70 protein (R&D System). Two different measurement times were used for the lower concentration range (500 ms) and high concentration range (100 ms). (A) Low ZAP-70 concentration in a range of 0-3,125 pg/reaction. (B) High ZAP-70 concentration in a range of 0-40,000 pg/reaction. The relationships at high concentration range are plotted on a log-log scale.

FIG. 8. Detection of ZAP-70 expression level in 20 peripheral blood samples from patients with CLL. The Y-axis displays normalized ZAP-70 signal (ratio of CLL cells-to-negative control cells, CLL/NC). The X-axis indicates blood sample numbers. The ZAP-70 results from flow cytometry analysis are showed below the chart. Samples from the same patients were also analyzed by flow cytometry assays developed by Food and Drug Administration (Degheidy H A et al., 2011a, Cytometry B Clin Cytom 80B:300-308; 2011b, Cytometry B Clin Cytom 80B:309-317). “+” means the patient was considered as having aggressive CLL. “−” means the patient was considered as having indolent CLL based on the method of Degheidy et al. 2011a,b. Genetic analysis showed patient P13 had mutations in IgVH (“M”). Patients P10 and P12 were found to have unmutated IgVH (“U”).

### DETAILED DESCRIPTION

### Abbreviations

TABLE 1
ABBREVIATIONS

| Abbreviation | Expansion |
|---|---|
| CLL | chronic lymphocytic leukemia |
| B-CLL | B cell CLL |
| ZAP-70 | zeta-chain-associated protein 70 |
| TCR | T-cell antigen receptor |
| BCR | B-cell antigen receptor |
| PBMC | peripheral blood mononuclear cell |
| IgVH | immunoglobulin variable heavy chain region gene |
| CD19 | cluster of differentiation 19 |
| ATCC | American Type Culture Collection |
| FITC | fluorescein isothiocyanate |
| Cy | cyanine |
| GST | glutathione-S-transferase |
| SH2 | Src homology 2 domain |
| PE | phycoerythrin |
| APC | allophycocyanin |
| PCR | polymerase chain reaction |
| RT-PCR | reverse transcriptase PCR |
| FACS | fluorescence-activated cell sorting |
| FI | fluorescence intensity |
| NFI | normalized fluorescence intensity |
| PC | positive control |
| NC | normal control |
| PBS | phosphate buffered saline |
| PBST | PBS containing 0.05% Tween-20 |
| Ex | excitation |
| Em | emission |
| ms | millisecond |
| LED | light-emitting diode |
| pg | picogram |
| r-ZAP-70 | recombinant ZAP-70 protein |
| JCL | Jurkat cell lysate |
| MACS | magnetic activated cell sorting |
| MPC | magnetic particle concentrator |

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims. Unless defined otherwise, all technical and scientific terms used herein have the same meaning as those commonly understood to one of ordinary skill in the art to which this invention pertains.

As used herein and in the claims, the singular forms “a,” “an,” and “the” include the plural reference and equivalents known to those skilled in the art unless the context clearly indicates otherwise. Other than in the operating examples, or where otherwise indicated, all numbers expressing quantities of ingredients or reaction conditions used herein should be understood as modified in all instances by the term “about.”

All patents and other publications identified are incorporated herein by reference for the purpose of describing and disclosing, for example, the methodologies described in such publications that might be used in connection with the present invention, but are not to provide definitions of terms inconsistent with those presented herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on information available to the applicants and do not constitute any admission as to the correctness of the dates or contents of these documents.

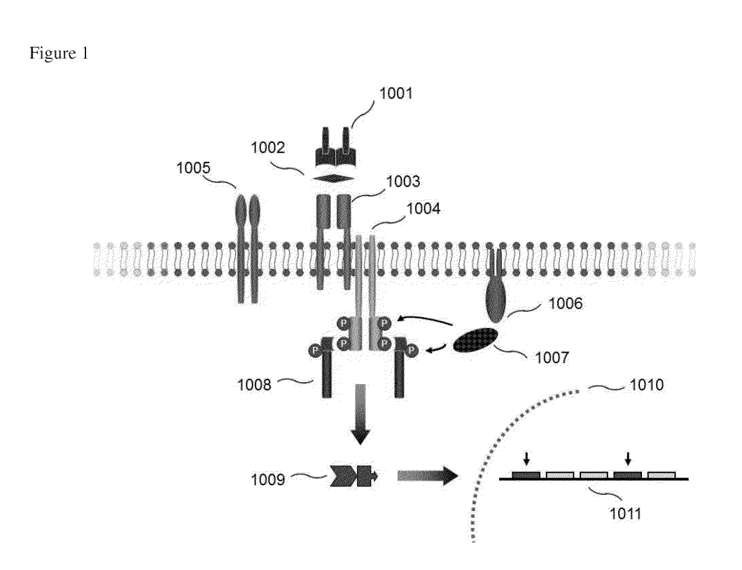

As suggested above, in one aspect of the first embodiment of the present invention, the invention is directed to methods for diagnosing chronic lymphocytic leukemia (CLL) in a subject based on the amount of ZAP-70 measured in a test sample. Similarly, and in one aspect of the second embodiment of the present invention, the invention is directed to immunoassays that can be used in the detection of ZAP-70 in a test sample. The location and biological function of ZAP-70 are depicted in FIG. 1. ZAP-70 1008 is a member of the protein tyrosine kinase family, and the protein is normally expressed in T cells and natural killer cells (Chan et al., 1991, Proc Natl Acad Sci USA 88:9166-9170; Vivier et al., 1993, Eur J Immunol 23:1872-1876). ZAP-70 plays a critical role in T-cell development and lymphocyte activation (Chan et al., 1992, Cell 71:649-662; Isakov et al., 1995, J Exp Med. 181:375-380; Au-Yeung et al., 2009, Immunol Rev 228:41-57). Activation of ZAP-70 involves MHC Class II 1001 and peptide 1002 from antigen presenting cells (e.g. macrophages, dendritic cells and B cells), CD3 1005, and T-cell antigen receptor (TCR) 1003. Upon signaling, CD4 1006 and the tyrosine kinase Lck 1007 become activated and phosphorylate the intracellular portions of the CD3 complex (called ITAMs). The most important member of the CD3 family is CD3-zeta 1004, to which ZAP-70 binds. ZAP-70 1008 is phosphorylated on tyrosine residues upon TCR stimulation, and the protein functions as a tyrosine kinase in the initial step of TCR-mediated signal transduction in combination with the Src family kinases (Kane et al., 2000, Curr Opin Immunol 12:242-249; Deindl et al., 2007, Cell 129:735-746). In turn, phosphorylated ZAP-70 interacts with adaptor proteins 1009, and sends messages to the nucleus 1010, where the transcription of several gene products 1011, which allow the T cells to differentiate, proliferate and secrete a number of cytokines, is activated.

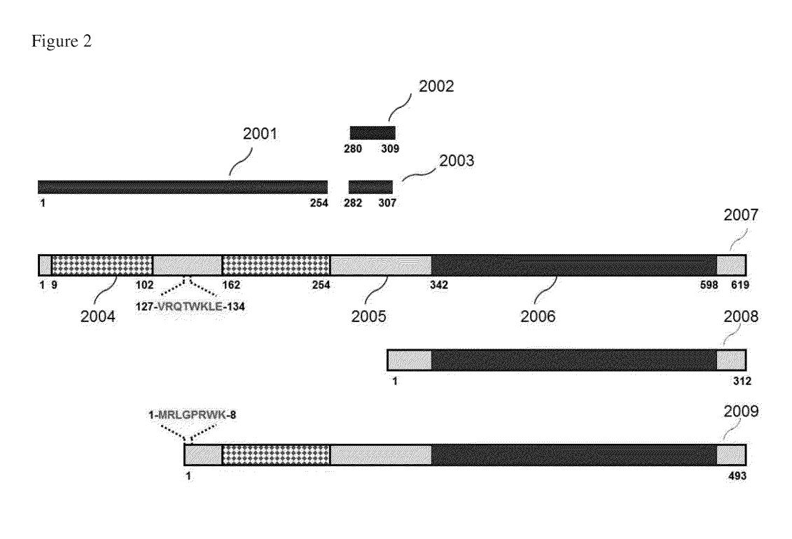

Three isoforms of ZAP-70 are depicted in FIG. 2. The full length of ZAP-70 protein 2007 is composed of three functional domains: two tandemly arranged Src homology 2 (SH2) domains 2004 at the N-terminus, a tyrosine kinase catalytic domain at the C-terminus 2006 and an inter-domain region 2005. Three isoforms with different amino acid sequences have been found for this protein (Strausberg et al., 2002, Proc Natl Acad Sci USA 99:16899-16903; Kuroyama et al., 2004, Biochem Biophys Res Commun. 315:935-941). The isoform-1 2007 contains a full-length protein of 619 amino acid sequences. The isoforms-2 2008 contains a short-length protein of only 312 amino acids with an N-terminal deletion from positions 1 to 307. The isoform-3 2009 contains 493 amino acids with an N-terminal deletion from 1-126. In addition, the first eight amino acids (VRQTWKLE; SEQ ID NO:1) of the isoform-3 are different from the isoform-1 at the corresponding positions 127-134 (MRLGPRWK; SEQ ID NO:2).

The methods of the present invention provide simple prognostic methods for diagnosing chronic lymphocytic leukemia (CLL) in a subject. Because of the greatly improved sensitivity of the methods over those currently available, the methods can also be used to distinguish between indolent and aggressive forms of CLL. The methods are based on determining the amount of ZAP-70 protein in a test sample obtained from a subject, such as a subject having CLL or suspected of having CLL. The determined amount of ZAP-70 is then compared to a standard, that is, the amount of ZAP-70 determined for a similar sample obtained from a single healthy control or a pool of healthy controls that are known to not have CLL. By making a quantitative determination of the amount of ZAP-70 in the test sample and comparing it to a standard, a diagnosis regarding CLL, or the subtype of CLL, can be made for the subject.

In a first specific aspect of the diagnostic methods of the invention, the method comprises preparing a ZAP-70 ratio M/N, wherein the ratio is prepared by quantitatively determining the amount of ZAP-70 M in a lysate of a test sample from a test subject, and dividing the determined amount M by a control amount N, wherein the control amount N is obtained by quantitatively determining the amount of ZAP-70 in a lysate of a control sample of the same identity from a control subject without CLL, wherein when the ZAP-70 ratio M/N is greater than about 2.0, the test subject is diagnosed as having an aggressive form of CLL, and wherein when the ZAP-70 ratio M/N is less than about 2.0 the test subject is diagnosed as having an indolent form of CLL. In a preferred example of this aspect, the test sample and the control sample are a predetermined number of B cells. In another preferred example of this aspect, the control amount N is obtained by quantitatively determining the amount of ZAP-70 in a lysate of a control sample of the same identity as the test sample from a pool of control subjects without CLL.

The ZAP-70 ratio is not affected by the ZAP-70 quantitation method as long as (i) the detection method is quantitative and linear, (ii) the dynamic range is large, and (iii) the standard of deviation is small compare to the signal. The explanation for this is as follows. The amount of ZAP-70 from a cell lysate from the test subject can be termed M and the amount of ZAP-70 from a cell lysate from the control subject can be termed N. The signal from the assay can be assumed to be linear and quantitative, and it is proportional to the amount of ZAP-70 as a. Furthermore, the standard deviation of the test subject is Δ, and the standard deviation of the control subject is δ. The amount of ZAP-70 from the cell lysate of the test subject is (aM+Δ). The amount of ZAP-70 from the cell lysate of the control subject is (aN+δ). The ratio of the quantitative amount of ZAP-70 of the test subject can be obtained by the following ratio.

(aM+Δ)/(aN+δ)≈M/N

In a second specific aspect of the diagnostic methods of the invention, the method comprises (a) preparing a normalized ZAP-70 value X, wherein the normalized value is prepared by (i) quantitatively determining the amount of ZAP-70 M in a lysate of a test sample from a test subject, (ii) quantitatively determining the amount of a control marker P in the same lysate, and (iii) dividing the amount determined for ZAP-70 by the amount determined for the control marker to obtain the normalized ZAP-70 value where X=M/P, (b) preparing a reference ZAP-70 value Y, wherein said reference value is prepared by (i) quantitatively determining the amount of ZAP-70 N in a lysate of a control sample of the same identity as the test sample from a control subject without CLL, (ii) quantitatively determining the amount of the control marker R in the same lysate, and (iii) dividing the amount determined for ZAP-70 by the amount determined for the control marker to obtain the reference ZAP-70 value where Y=N/R, and (c) obtaining a ZAP-70 ratio X/Y by dividing the normalized ZAP-70 value X by the reference ZAP-70 value Y, wherein when the ZAP-70 ratio X/Y is greater than about 2.0, the test subject is diagnosed as having an aggressive form of CLL, and wherein when the ZAP-70 ratio X/Y is less than about 2.0 the test subject is diagnosed as having an indolent form of CLL. In a preferred example of this aspect, the test sample and the control sample are a predetermined number of B cells. In another preferred example of this aspect, the lysate of a control sample of the same identity as the test sample is from a pool of control subjects without CLL.

In this second specific aspect of the diagnostic methods of the invention, only one assay needs to be performed wherein both the amount of ZAP-70 and the amount of the control marker can be determined at the same time. The reference ZAP-70 value N and the control marker value R can be obtained beforehand and retrieved for reference. For example, two different fluorescence dyes can be used to quantitate two markers in the same assay. In some cases, the assay data will be most useful when the amount of the control marker P is the same as the amount of control marker R. Again, the ZAP-70 ratio is not affected by the ZAP-70 quantitation method as long as (i) the detection method is quantitative and linear, (ii) the dynamic range is large, and (iii) the standard of deviation is small compare to the signal.

In the second specific aspect of the diagnostic methods, the control marker may be any marker that does not vary based on whether the subject has CLL. In preferred aspects, the control marker a housekeeping protein, such as a tyrosine kinase protein (e.g. Syk), a human leukocyte differentiation antigen (e.g. CD45), and a transmembrane receptor protein, such as B-cell receptor (BCR), etc. The skilled artisan will understand that the identity of the control marker will be the same in each step of the diagnostic methods of the invention. That is, for example, the control marker of (a)(ii) and (b)(ii) in the second specific aspect of the diagnostic methods of the invention provided above will be the same.

In each aspect of the methods of the invention, the test and control samples are any biological material that will contain the protein to be assayed. For example, the samples may be, but are not limited to, a biological fluid, a population of cells, a cell lysate, a tissue, and bone marrow. In certain aspects, the biological fluid is blood, plasma, serum, spinal fluid, saliva, urine, tears, or mucus. In certain aspects, the population of cells is a predetermined number of: B cells, T cells, tumor cells, fetal cells, epithelial cells, or blood cells. In one particular aspect, the sample is a predetermined number of B cells. By using a predetermined number of cells, such as B cells, the method can easily be standardized, allowing a standard curve to be made from a control subject or pool of control subjects not having CLL well in advance of assaying of a population of cells from a subject having CLL or suspected of having CLL. Indeed, in each aspect of the methods of the invention, the test samples and control samples used in the comparisons must be as close in identity as possible, to ensure that the comparison is as relevant as possible. Identity should be present in such characteristics as the number of cells in the sample, the source of the sample, the means by which the sample is processed, and the age, sex and general health of the subjects from which the samples are obtained.

The control subject or pool of control subjects not having CLL is preferably healthy subjects. In particular, it is important for the amount of ZAP-70 in the control subjects to reflect the level found in subjects without CLL. Because the amount of ZAP-70 in the B cells of subjects having other illnesses and diseases has not been surveyed, using control subjects otherwise free of any illness or disease will help to ensure that their B cells express “CLL-free” amounts of ZAP-70.

The diagnostic methods of the invention generally provide a diagnosis of an aggressive form of CLL when the ZAP-70 ratio is greater than about 2.0, and a diagnosis of an indolent form of CLL when the ZAP-70 ratio is less than about 2.0. However, the skilled artisan will understand that values slight lower than about 2.0 can also be the basis for a diagnosis of an aggressive form of CLL, such as about 1.5, about 1.6, about 1.7, about 1.8 or about 1.9. Under these circumstances, other factors may be taken into consideration in the diagnosis. Similarly, the skilled artisan will understand that values slight higher than about 2.0 can also be the basis for a diagnosis of an indolent form of CLL, such as about 2.1, about 2.2, about 2.3, about 2.4 or about 2.5. Under these circumstances, other factors may again be taken into consideration in the diagnosis.

The amount of ZAP-70 and/or control marker in a lysate of a test sample is determined by four general steps:

a) collecting a test sample or control sample from the subject,

b) lysing the cells in the sample,

c) capturing the ZAP-70 and/or control marker from the cell lysate of b), and

d) measuring the amount of ZAP-70 and/or control marker captured in c).

These steps are exemplified with respect to a blood sample in the following sections. The blood can be any peripheral blood or bone marrow sample, such as those that are collected in vacuum tubes containing an anti-coagulant, such as EDTA, heparin or acid-citrate-dextrose. The sample can be also any culture cell lines. In a preferred embodiment, the sample is collected in a K2-EDTA tube where the sample is peripheral blood (usually in a range of approximately 8-10 mL) that is obtained from the subject.

In order to reduce the amount of potentially cross-reactive species in a sample, a selected population of cells, such as B cells, is preferably isolated from the test sample for further processing. Further, by isolating a predetermined population of cells, one may also easily standardize the diagnostic method by ensuring that the same number of cells is utilized each time the method is performed. For example, when a predetermined number of B cells are utilized in the method, B cells may be removed from blood obtained from the subject.

1. Separation of B Cells from the Blood Sample

A variety of methods can be used for separation of B cells including but not limited to separations based on immunomagnetic separation (IMS) using magnetic beads coated with antibody against B cells, Ficoll gradient centrifugation, depletion of non-B cells (negative selection), magnetic activated cell sorting (MACS, Miltenyi CD19 microbeads), fluorescence-activated cell sorting (FACS), and other immune-separation techniques, etc. One preferred method for separation of B cells is use of Ficoll gradient centrifugation followed by immunomagnetic separation. Another preferred method is immunomagnetic separation directly using whole blood.

The separation of B cells can be accomplished by using Ficoll gradient centrifugation followed by immunocapture using microbeads coated with antibody specific to B cell surface markers (e.g. CD19, CD38 and other markers). The beads can be nanoparticles, magnetic beads, solid glass beads, hollow glass beads, polystyrene latex beads or other microspheres. These B cell-specific capture materials can be monoclonal antibodies, polyclonal antibodies, aptamers, etc. Examples of antibodies are antibodies that recognizes CD19+ antigen on the surface markers of B cells.

The separation of B cells can also be accomplished by immunocapture using microbeads coated with antibody specific to B cell surface markers directly from whole blood. The MAC beads are efficient.

Another method to collect the B cells is negative selection. The B cells in the sample are untouched but all of the other cells are depleted and removed by specific capture, leaving only B cells. One method is red blood cell lysis. Following the depletion, the B cells can be collected and concentrated by non-specific methods, such as filtration and centrifugation, or by specific methods, such as antibody capture of B cells. Furthermore, B cells can be obtained by FACS or by any other methods used for purification of B cells.

Once a population of B cells is isolated from blood, the cells can be divided into aliquots of a known number, where the aliquots serve as predetermined numbers of B cells for use in the further steps in the method.

### 2. Preparation of Cell Lysates

As suggested above, the present invention is directed in a second embodiment to immunoassays that can be used in the detection of selected proteins in a test sample. The immunoassay can be used separately or in conjunction with the diagnostic methods of the present invention. The immunoassay enables the rapid and efficient detection and quantification of a biomarker in a test sample, such as blood. The immunoassay comprises steps b) through d) noted above, namely: b) lysing the cells in the test or control sample, c) capturing the protein from the cell lysate of b), and d) measuring the amount of protein captured in c).

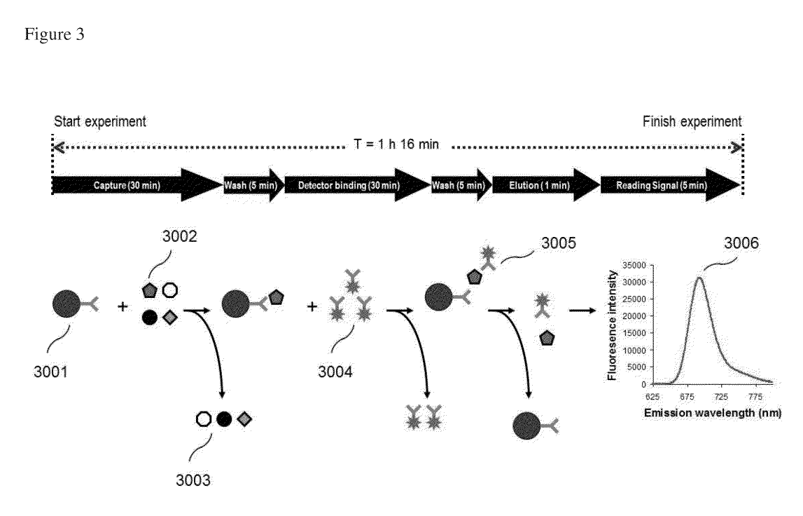

FIG. 3 provides a schematic diagram illustrating general principles of one means for using the immunoassay in the detection ZAP-70 in a cell lysate. An anti-ZAP-70 capture antibody is first immobilized on the magnetic beads 3001. The ZAP-70 protein 3002 in the cell lysate is specifically captured by the capture antibody-bound magnetic beads. Other unbound proteins and contaminates 3003 are washed away. A fluorescent detector antibody 3004 recognizes the bead-captured ZAP-70, forming an immunosandwich complex 3005. Unbound detector antibodies are washed away. The detector antibody is further eluted from the magnetic beads. Distinct fluorescence signal 3006 of the detector antibody, corresponding to the ZAP-70 concentration, is measured using a quantitative and sensitive fluorescence detection instrument, for example a Signalyte™-II spectrofluorometer. By comparison of fluorescence intensities from test sample and control sample, the results can be reported in quantitative format. The immunoassay is exemplified in the following sections using ZAP-70 as the target.

The test and control samples are preferably in the form of a solution or suspension of the target cells, such as a predetermined numbers of B cells that can be processed directly in the assay. Thus, in one embodiment of the invention, the immunoassay system enables detection of the ZAP-70 protein rather than detection of whole B cells that express the protein. A variety of methods can be used for preparation of cell lysates including but not limited to chemical cell lysis (cell lysis buffer), mechanical disruption, liquid homogenization, high frequency sound waves (sonication), freeze/thaw cycles and manual grinding. A preferred method uses cell lysis buffer (such as The Cell Lysis Buffer from Cell Signalying) followed by sonication. These cell lysis buffers contain a cocktail of protease inhibitors to control undesirable proteolysis. Since many of these compounds are not very stable in aqueous solutions so that they can maintain the stability of ZAP-70. The cell lysates can be stored at −20° C. condition for several months until use without loss of antigenic activity of ZAP-70.

The method of the invention enables elimination of several cell-treatment steps that are required in flow cytometry analysis. Flow cytometry is currently the most common method of choice for analysis of ZAP-70 expression in CLL cells. There are, however, some severe limitations that confound the use of flow cytometry as a routine diagnostic tool in clinical laboratories. Flow cytometry analysis requires red blood cell lysis, cell fixation and permeabilization to allow access of the fluorescent antibody to subcellular structures. These steps not only increase the complexity of the assay procedure, but also cause significant variations from laboratory to laboratory. In addition, the use of different antibody clones in flow cytometry can produce conflicting results for an individual patient. This discrepancy can be explained by varying accessibility of antibody to different epitopes and subcellular locations of the ZAP-70 protein. In human epithelial and T cells, a large amount of ZAP-70 resides in the nucleus of quiescent and activated cells. Therefore, free access of antibody to all subcellular locations is critical to detect both cytoplasmic and nucleic ZAP-70. The variations and discrepancies in flow cytometry analysis of ZAP-70 may be attributed to detrimental effects of fixation and permeabilization, such as fixation-induced masking of antigenic sites or insufficient accessibility of detector antibody to nucleic ZAP-70.

This invention overcomes the drawbacks of the flow cytometry method, because it directly detects ZAP-70 protein, rather than counting cells with detectable levels of the marker. There is no need of red blood cell lysis, cell fixation, and permeabilization. The method of the invention is designed for use of whole cell lysis to effectively release all cytoplasmic and nucleic ZAP-70 protein to a homologous supernatant. The interactions between ZAP-70 and anti-ZAP-70 antibody is uninhibited in the supernatant, allowing maximum access and binding of antibody to the target antigen. Subsequently, ZAP-70 protein is specifically captured on the surface of an antibody carrier, such as magnetic beads, and unbound proteins and other contaminants are washed away. Without this isolation of ZAP-70 by capture antibody, other proteins and contaminants in the system may interfere with assay sensitivity and specificity. Use of antibody-coated carriers, such as magnetic beads, eliminates the need to purify ZAP-70 from complicated mixtures before measurement, simplifying the assay and increasing the specificity and the sensitivity. Thus, interference from other cell components is minimized. With this assay format, it was possible to increase detection sensitivity and reduce inter-laboratory variation. The examples below demonstrate one implementation of the VeriZAP™ assay system. The protocol described in FIG. 3 and fluorescence detection using Signalyte™-II spectrofluorometer is a very sensitive assay that can detect ZAP-70 at levels lower than 39 pg of purified ZAP-70 protein or 125 Jurkat cells. This is much more sensitive than all the existing methods.

### 3. Protein Capture

An optimized ZAP-70 capture method is described. The invention provides high assay specificity in capturing protein from the cell lysate by using two specific antibodies (capture and detector antibodies) to recognize ZAP-70 in the lysate, and form an immunosandwich complex on the surface of a carrier, such as a magnetic bead. This requires that each ZAP-70 molecule contains at least two antigenic sites capable of binding to antibody in the sandwich format. As an example, clones 2F3.2/1E7.2 are selected as a capture/detector antibody pair. However, the antibodies used for this ZAP-70 assay are not limited to these two clones. Other clones of anti-ZAP-70 antibody can be used and optimized. The two clones antibodies, used as an example in this invention, bind ZAP-70 at two distinct sites. The clone 2F3.2 antibody was generated against GST-fusion protein containing the 2 tandem SH2 domains of human ZAP-70, corresponding to amino acids 1-254. The specific binding sites of this clone have not been mapped on the ZAP-70 amino acid sequences yet. If the binding sites located at the regions associated with the SH2 domains, use of clone 2F3.2 may pull down as much of the ZAP-70 as possible. The clone 2F3.2 is the antibody most commonly used in flow cytometry and immunohistochemistry for detection of ZAP-70. Our results further supported that the clone 2F3.2 was an effective antibody for capture of ZAP-70 in cell lysate samples. The clone 1E7.2 antibody was generated against a KLH-peptide sequence corresponding to the human ZAP-70 amino acids 282-307. As 1E7.2 recognizes a single epitope, it allows specific detection and quantification of small differences in antigen. Our data showed that this clone worked well as detector antibody when conjugated with either PE-Cy5.5 or Dylight 649. Although three isoforms of ZAP-70 have been reported, little is known about their expression and distribution in different cell types. According to the known antibody binding sites, the selected antibody pair of 2F3.2/1E7.2 could detect isoforms 1 and 3, but might not be efficient to detect isoform 2, a truncated version of ZAP-70. If there is any truncated ZAP-70 proteins in the test sample, additional antibody can be included in the assay to further improve assay efficiency.

In additional to antibodies, other molecules that specifically recognize and bind to the target protein (e.g., ZAP-70) may be used, including apatmers and ligands.

The antibodies, apatmers and ligands used in the immunoassays of the present invention are preferably attached to the surface of a carrier. Exemplary carriers include magnetic beads, glass beads, ferrofluids and microfluidic chips. Thus, specific examples of conjugates that may be used to capture the target protein (e.g., ZAP-70) from the lysate include the following: capture antibody-coated magnetic beads, aptamer-coated magnetic beads, ligand-coated magnetic beads, capture antibody-coated glass beads, aptamer-coated glass beads, ligand-coated glass beads, a capture antibody-containing ferrofluid, an aptamer-containing ferrofluid, a ligand-containing ferrofluid, a capture antibody-coated microfluidic chip, an aptamer-coated microfluidic chip and a ligand-coated microfluidic chip.

Once captured from the lysate, such as by a capture antibody-coated magnetic bead, the target protein (e.g., ZAP-70) can be detecting using a detector antibody labeled with a detectable label. As suggested above, the detector antibody binds to a different site on the protein than the capture antibody, allowing formation of an immunosandwich.

The detectable label may be a fluorescent dye, a dye particle or quantum dots. Various fluorescent dyes can be used in the method of the present invention. The fluorescent dye is selected depending on the target proteins, and the concentration of the target protein in the solution. The fluorescence dye is conjugated with the detector antibody. As an example, a defined number of B cells are used in VeriZAP™ assay to normalize the input cells. The normalization of input sample can be also achieved by using an internal control protein (e.g. a housekeeping protein with stable expression in B cells). By comparing ZAP-70 protein with the internal control, a ratio of ZAP-70 can be reported. The detector antibodies, targeting ZAP-70 and the internal control, can be conjugated with different fluorescence dyes, allowing that different targets can be detected in a format of multiplex assay. It has been found that fluorescent dyes that fluoresce in the red and far infrared range are particularly suitable for certain applications. The fluorescent dyes whose fluorescent emissions do not significantly overlap the Raman emission of water provide good sensitivity in the fluorometer. Raman emission of water introduces high background. For example, TexasRed (sulforhodamine 101 acid chloride), absorbing at 589 nm and emitting at 615 nm, and Cy™-5 and similar dyes, absorbing at 635 nm and emitting at 670 nm, are suitable. Additional examples of suitable dyes are DyLight series of dyes 638/658, 654/673, 692/712 excitation/emission wavelength in nm, and Alexa Fluor series of dyes 590/617, 612/626, 632/647, 633/647, 650/665, 663/690 excitation/emission wavelength in nm. Other long wavelength fluorescent dyes can also be used. In some applications, the short wave length fluorescent dyes are suitable but may not provide the sensitivity to exhibit rapid detection. In addition to fluorescence dyes, other labeling tags, such as horseradish peroxidase (HRP), alkaline phosphatase (AKP), dye particles, or quantum dots (QD), etc. can be also conjugated with the detector antibodies for use in the method of the invention.

### 4. Measurement of ZAP-70 Signal

One aspect of the immunoassays of the present invention comprises detection of fluorescence signal from ZAP-70 in a liquid solution. To achieve this, the immunosandwich complex is dissociated from the carrier (e.g., magnetic beads) and the supernatant containing the complex is used for the fluorescence signal detection. This eliminates the background noise from the carrier so that more specific and reproducible results can be obtained. Only the ZAP-70 signal, which is specifically produced by the detector antibody, will be measured. The measurement of ZAP-70 signal (i.e., the label of the detector antibody) can be accomplished using an ultra-sensitive spectrofluorometer, such as Signalyte™-II. This spectrofluorometer was developed by Creatv MicroTech based on the detection principles of Integrating Waveguide Technology (U.S. Pat. No. 7,801,394, issued Sep. 21, 2010). However, the instrument for measurement of ZAP-70 signals is not limited to Signalyte™-II. Other spectrofluorometers and fluorometers, such as standard fluorometers, microplate fluorometers or NanoDrop Fluorospectrometer, etc., can be also used for measurement of ZAP-70 signals.

Signalyte™-II is a preferred instrument because it offers advantages of high sensitivity, large dynamic range, appropriate sample size and quantitative results. Signalyte™-II uses proprietary liquid phase integrating waveguide technology to measure fluorescence signal in liquid samples. Eight test samples and one control sample can be measured in a tube holder. The principle of detection consists of illuminating the cuvette at a 90° angle relative to the length of the waveguide and subsequent collection of the emitted fluorescence from the sealed end. The emitted light is efficiently gathered by the cuvette and guided by the cuvette/sample as a waveguide. The emission signal exits from the end of the waveguide, through lenses and filters to the optical detector. Emitted light from the entire waveguide is integrated, thus maximizing the signal collection. Background noise from the excitation light is minimized by the 90° excitation angle. Signal-to-noise ratio is improved compared to other test geometries, enabling more sensitive assays. The instrument can test eight samples, plus one control in about one minute. Illumination wavelengths are available from 365 nm to 635 nm to excite most organic dyes and quantum dots. This is achieved by a combination of high power LEDs and bandpass filters. The detector in Signalyte™-II is a spectrometer that detects emissions in a range of 350 nm to 800 nm. This spectral range is suitable for the most common fluorescence-based applications.

The present invention is exemplified with respect to ZAP-70 marker in CLL. However, it can be used for any disease in which a protein is a biomarker for prognostics or diagnostics, including other types of leukemia or lymphoma, infectious diseases, cancers, genetic diseases and autoimmune diseases, etc.

The following examples are provided to facilitate a thorough understanding of the invention for purposes of explanation, but not limitation. The specific details set forth particular embodiments of the VeriZAP™ assay system but are also applicable to the general immunoassays of the present invention. Thus, the invention can be practiced in other embodiments that depart from these specific details.

### Example I

### Preparations of Assay Reagents, Controls and Cell Lysates

Preparations of Positive Control, Antibodies and Reagents.

The immunoassay is based on quantitative, rather than qualitative, analysis. Improvements include use of controls of ZAP-70-positive cells (Jurkat cells) and ZAP-70-negative cells (normal B cells). In particular, purified recombinant ZAP-70 protein is included as target molecule control for validation and construction of standard curve. Human lymphocyte-derived cell lines can be obtained from the American Type Culture Collection (ATCC). One cell line, commonly used as ZAP-70 positive control, is the E6-1 clone of wild-type Jurkat cells (ATCC No. TIB-152). This cell line expresses ZAP-70 protein and serves as a positive control in the methods of the invention. The cell line was grown in the complete growth medium of RPMI-1640 according to ATCC protocols. All antibodies and reagents useful in the methods of invention are listed in Table 2. The biotinylated antibodies, fluorescence-conjugated antibodies and immunoaffinity magnetic beads were prepared using standard protocols as previously described (Zhu et al., 2005, Biosens Bioelectron 21:678-683; 2011, Biosens Bioelectron 30:337-341).

TABLE 2
CELL LINE, ANTIBODIES AND REAGENTS

| Materials | Catalogue No. | Supplier | Location | Description | |
|---|---|---|---|---|---|
| Jurkat cell line (clone E6-1) | TIB-152 | ATCC | Manassas, VA | 1 | vial/PK |
| Jurkat cell lysate | 12-303 | Millipore | Billerica, MA | 1 | mg/PK |
| Recombinant human active | 3709-KS | R&D Systems | Minneapolis, MN | 10 | μg/PK |
| ZAP-70 | | | | | |
| Anti-ZAP-70 (human) antibody, | 05-253MG | Millipore | Billerica, MA | 1 | mg/PK |
| clone 2F3.2 | | | | | |
| Mouse anti-human ZAP-70 | 731893 | Beckman Coulter | Fullerton, CA | 0.1 | mg/PK |
| antibody, clone SBZAP | | | | | |
| Anti-mouse/human ZAP-70 | 14-6695 | eBioscience | San Diego, CA | 0.1 | mg/PK |
| antibody, clone 1E7.2 | | | | | |
| Anti-ZAP-70 APC (clone 1E7.2) | 17-6695 | eBioscience | San Diego, CA | 0.5 | μg/test |
| Anti-ZAP-70 PE-Cy5.5 (clone | 35-6695 | eBioscience | San Diego, CA | 0.2 | mg/ml |
| 1E7.2) | | | | | |
| PE-anti-human CD20 | 12-0209 | eBioscience | San Diego, CA | 0.06 | μg/test |
| APC-anti-human CD3 | 17-0038 | eBioscience | San Diego, CA | 0.25 | μg/test |
| FITC-anti-human CD19 | 302205 | BioLegend | San Diego, CA | 25 | tests/PK |
| Human CD3 Microbeads | 130-050-101 | Miltenyi biotec | Auburn, CA | 2 | ml/PK |
| Human CD19 Microbeads | 130-050-301 | Miltenyi biotec | Auburn, CA | 2 | ml/PK |
| BCA protein assay kit | 23225 | Pierce | Rockford, IL | 500 | tests/PK |
| Cell lysis buffer (10×) | 9803 | Cell Signaling | Danvers, MA | 15 | ml/PK |
| Dylight 649 NHS Ester | 46416 | Thermo Scientific | Rockford, IL | 5 × 50 | μg/PK |
| Cy5 mAb labeling kit | PA35001 | GE Healthcare | Piscataway, NJ | 5 | reactions/PK |
| Ficoll-Paque PLUS | 17-1440-02 | GE Healthcare | Piscataway, NJ | 6 × 100 | ml/PK |
| Zeba spin desalting column | 89889 | Thermo Scientific | Rockford, IL | 6 | columns/PK |
| Dynabeads M-280 Streptavidin | 112.06D | Invitrogen | Carlsbad, CA | 10 | ml/PK |
| EZ-Link Sulfo-NHS-LC-Biotin | 21327 | Thermo Scientific | Rockford, IL | 8 × 1 | mg/PK |

Processes of Human Whole Blood Samples and Preparations of T and B Cells.

Peripheral blood samples were obtained from patients with CLL and healthy blood donors. Viable PBMCs were isolated from 12 ml of blood sample by density gradient centrifugation using Ficoll-Paque Plus (GE Healthcare, Piscataway, N.J.). The recovered PBMCs were counted with hemocytometer and adjusted to a final concentration of 10⁷ cells/ml. The B cells were isolated from PBMCs by MACS system using Human CD19 MicroBeads Systems (Miltenyi, Auburn, Calif.) according to the manufacture's instructions, while the T cells were isolated from PBMCs by MACS system using Human CD3 MicroBeads Systems (Miltenyi). The T cells were used as one of the assay controls. These purified cells were used for preparation of cell lysate or stored at −80° C. in cryo-fluid until use. The purity of the isolated B cells was confirmed by staining with fluorescent antibodies and flow cytometry analysis. Briefly, the cells were fixed in 1% paraformaldehyde-saline solution and stained with FITC-labeled anti-CD19 antibody to identify B cells (BioLegend), APC-labeled anti-CD3 antibody to identify T cells (eBioscience) and PE-labeled anti-CD20 antibody also to identify B cells (eBioscience). This flow cytometry analysis showed that the B cell purity in the preparations was >98%.

Preparation of Cell Lysates.

The purified B cells were counted with a hemocytometer. To prepare cell lysate, one million of the purified B cells were suspended in 200 μl of 1× Cell Lysis Buffer and incubated on ice for 5 min. The lysate was sonicated in a water-bath sonicator (Model 450 ultrasonic cleaner, E/MC Corp.) for 1 min. The lysate sample was heated at 95° C. in a water bath for 5 min, placed on ice for 1 min, and then centrifuged at 12,000×g for 1 min to bring down all liquid evaporates. The lysate was then transferred to a QIAamp Mini Column (Qiagen) and centrifuged at 12,000×g for 2 min to remove genomic DNA and cell debris. The pass-through fraction in the collection tube (the final cell lysate sample) was stored at −20° C. until use for further analysis.

Illustration of the Assay Principle for Detection of ZAP-70 in the Cell Lysate.

Expression of ZAP-70 protein in lymphocytes was assessed by the protocol illustrated in FIG. 3. VeriZAP™ assay consists of collecting and lysing a defined number of leukemic cells followed by detection of ZAP-70 proteins in the cell lysates. The detection of ZAP-70 consists of two basic reaction steps: immunomagnetic separation of ZAP-70 and fluorescence antibody detection of ZAP-70. (i) Immunomagnetic separation. The assay principle of the invention is depicted in FIG. 3. A test sample was prepared in a 1.5 ml Eppendorf tube by mixing 10 μl of cell lysate sample with 990 μl of PBS. Five μl of anti-ZAP-70 immunomagnetic beads 3001 was added in the sample. The mixture was incubated on a Labquake rotator (Barnstead Thermolyne, Melrose Park, Ill.) with rotation at room temperature for 30 min, allowing the magnetic beads to capture the ZAP-70 present in the sample 3002. The beads were separated by placing the tube on the Dynal MPC-S rack (Invitrogen) for 2 min. The supernatant, containing the unbound components and other contamination 3003, was removed. The beads were then washed three times as follows: the beads were resuspended in 1 ml of PBS containing 0.05% tween-20 (PBST); each tube was inverted gently 4-5 times and then placed in MPC-S for 2 min. Following the final wash, supernatant was removed and the beads were ready for fluorescence antibody detection 3004. (ii) Fluorescence antibody detection. 100 μl of 10 μg/ml PE-Cy5.5-conjugated anti-ZAP-70 antibody (clone 1E7.2) was added to the beads and incubated on a Thermomixer R (Eppendorf) at 37° C. for 30 min with 200 rpm shaking. The beads were recovered by MPC-S and washed four times with PBST to remove unbound antibody. To dissociate the immunocomplex 3005, the beads were suspended in 100 μl of Elution Buffer (Creatv) by vortexing for 30 sec and incubated at room temperature for 2 min, and vortexed again for another 30 sec. The bead vial was placed on MPC-S rack for 2 min. 40 μl of supernatant was transferred to a glass capillary (Roche Diagnostics, Indianapolis, Ind.). The fluorescence intensity (FI) 3006 of PE-Cy5.5 was measured with Creatv's Signalyte™-II with excitation and emission wavelengths of 650 nm and 692 nm, respectively. Elution Buffer was used as reference to subtract the background and obtain normalized fluorescence intensity (NFI): NFI=FI_{Sample}−FI_{reference}.

### Example II

### Selection of Antibodies and Fluorescence Dyes

Selection of Anti-ZAP-70 Antibodies.

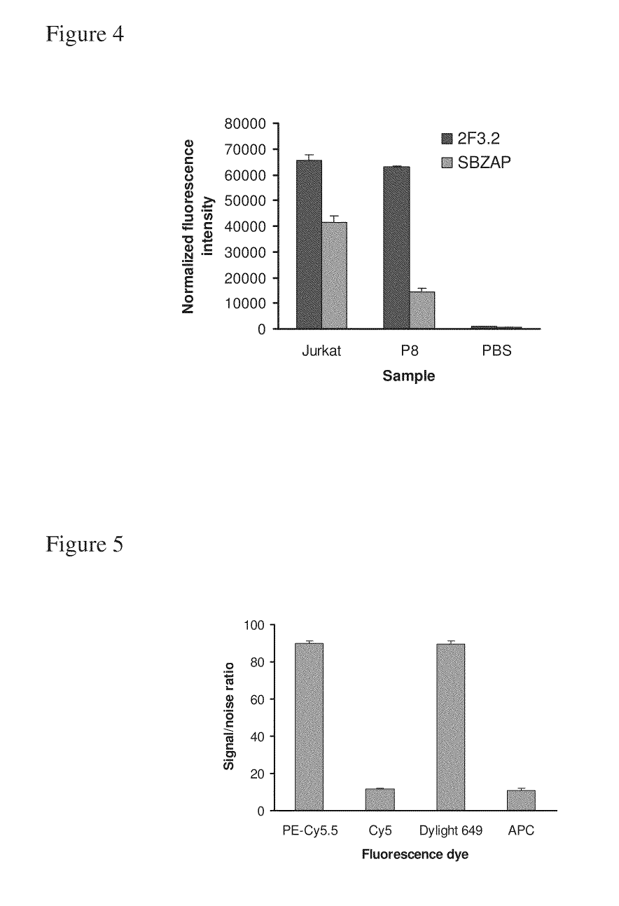

In this experiment, three different anti-ZAP-70 monoclonal antibodies were evaluated for detection of ZAP-70 in this study (clones 2F3.2, SBZAP and 1E7.2). The locations of the immunogens used to generate these antibodies are shown in FIG. 2. The clones 2F3.2 and SBZAP were used as a capture antibody, whereas 1E7.2 was used as a detector antibody, forming two combinations of antibody pairs: 2F3.2/1E7.2 and SBZAP/1E7.2. The capture antibody 2F3.2 was immobilized on the surface of magnetic beads through biotin/avidin interaction. Phycoerythrin-Cy5.5 (PE-Cy5.5)-conjugated 1E7.2 (eBioscience) was used as the detector antibody. Two ZAP-70-positive controls, Jurkat cell lysate and CLL cell lysate (P8) were tested with these antibody pairs. The results showed that antibody pair 2F3.2/1E7.2 produced higher fluorescence signal than antibody pair SBZAP/1E7.2 for Jurkat cell lysate (1.6-fold) and for CLL cell lysate (4.3-fold) (FIG. 4). Both antibody pairs were highly specific to ZAP-70, as demonstrated by a very low fluorescence signal for the negative control. In addition, use of 1E7.2 as capture antibody did not improve the final signal (data not shown), suggesting that 2F3.2 was a more suitable antibody than 1E7.2 for the capture reaction. The antibody pair 2F3.2/1E7.2 showed better sensitivity and was selected for detection of ZAP-70 protein in the following experiments.

Selection of Fluorescence Dyes.

In order to minimize non-specific background signal, four fluorescence-conjugated 1E7.2 antibodies were evaluated in this study. The PE-Cy5.5- and allophycocyanin (APC)-conjugated 1E7.2 antibodies were obtained from eBioscience. Cy5- and Dylight 649-conjugated 1E7.2 antibodies were prepared in house. Jurkat cell lysate and PBS buffer were used as positive and negative controls, respectively. For Jurkat cell lysate, the fluorescence signal was determined as Cy5-1E7.2 (51,014)>Dylight 649-1E7.2 (17,007)>PE-Cy5.5-1E7.2 (5,995)>APC-1E7.2 (97). The signal-to-noise (S/N) ratio of the Jurkat cell lysate to the PBS control was determined as PE-Cy5.5-1E7.2 (90.1)>Dylight 649-1E7.2 (89.8)>Cy5-1E7.2 (11.78)>APC-1E7.2 (10.7) (FIG. 5). Although Cy5-conjugated antibody produced the highest fluorescence signal, its background noise was also higher, which significantly reduced the S/N ratio for this dye. PE-Cy5.5- and Dylight 649-conjugated antibodies produced a relatively high signal for ZAP-70 and low background, yielding the highest S/N ratio. PE-Cy5.5-conjugated antibody could be excited by Signalyte™-II at two different wavelengths, either 470 nm or 635 nm, to produce an emission peak at 692 nm. Although the detection sensitivity was similar for the two excitation wavelengths, excitation at 635 nm produced better linearity at high concentrations of ZAP-70. Based on these results, we selected PE-Cy5.5-1E7.2 antibody as the detector antibody and used excitation/emission wavelengths at 635/692 nm for measurement of ZAP-70 signals.

### Example III

### Detection of ZAP-70 in Jurkat Cell Lysates

Detection of ZAP-70 in Jurkat Cell Lysate.

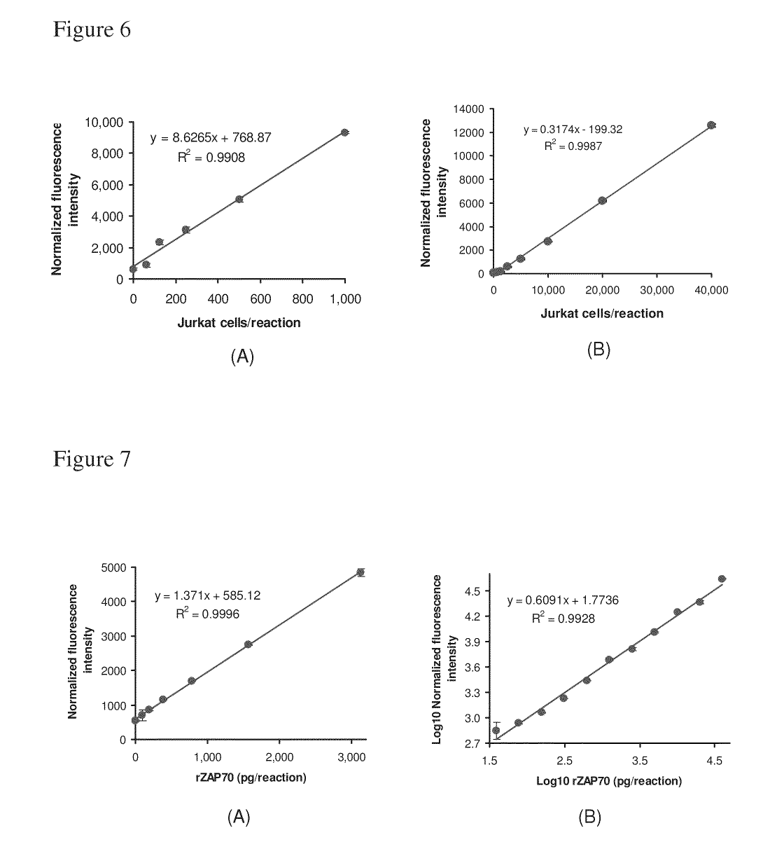

Jurkat cell lysate, the most common reference for ZAP-70, was used for evaluation of the method of the invention. To determine the detection sensitivity, two-fold serial dilutions of Jurkat cell lysate were prepared with a dilution solution (PBS containing 2% lysis buffer) to obtain final concentrations ranging from 63-1,000 cells/reaction. These serial dilutions were tested using PE-Cy5.5-conjugated detector antibody. Fluorescence signal was measured with excitation/emission wavelengths (Ex/Em) at 635/692 nm. Fluorescence intensity (FI) of each sample was measured with Signalyte™-II. In order to obtain a very low limit of detection, it was necessary to normalize the FI signal by subtracting the FI of a buffer control from the FI of each sample. This resulted in the normalized fluorescence intensity (NFI) of each sample. The relationship between NFI and Jurkat cell concentration is plotted in FIG. 6A, showing a strong linear relationship (R²=0.9808). The equation describing the relationship is y=8.6265x+768.87. Based on average NFI and standard deviation (S.D.) of the negative control (79±53.74), a threshold NFI value (average+3S.D.) was set at 240.22. The input concentration of 125 cells resulted in a significantly higher signal (395.5±10.61) than that of the negative control. Thus, limit of detection of ZAP-70 in Jurkat cell lysate was determined to be lower than 125 cells. To determine the assay dynamic range, serial dilutions including higher concentrations (up to 100,000 cells/reaction) were tested. To avoid signal saturation at high cell concentrations, a shorter excitation time (10 ms, equivalent to 1/50 in FIG. 6A) was used for measurement of fluorescence signals. The relationship between NFI and higher concentrations of the Jurkat cells is plotted in FIG. 6B. A linear signal response was observed over a wide range of 63-40,000 cells/reaction (R²=0.9987).

### Example IV

### Detection of Recombinant ZAP-70 Protein

Detection of Recombinant ZAP-70 Protein.

Purified recombinant ZAP-70 proteins (r-ZAP-70) from three R&D System was used as positive controls in serial dilution tests and spiking experiments to validate assay specificity. The results showed that the r-ZAP-70 protein from R&D System produced the best sensitivity. A strong linear relationship (R²=0.99996) between NFI and r-ZAP-70 concentrations was observed from 0-3,125 pg/reaction (FIG. 7). The equation describing the relationship is y=1.371x+585.12. Based on average NFI and S.D. of the negative control (533.50±24.75), a threshold NFI value was set at 607.75. The input concentration of 39 pg/reaction resulted in a significantly higher signal (697.5±157.68) than that of the negative control (FIG. 7). The LOD for r-ZAP-70 of R&D System was thus determined to be lower than 39 pg of the r-ZAP-70 per reaction. In addition, the relationship between NFI and higher concentrations of r-ZAP-70 was plotted on a log-log scale. A linear signal response was observed over a wide range of 0-40,000 pg/reaction (R²=0.9928). Overall, these results demonstrate that the method of the invention is extremely sensitive and specific for detection of ZAP-70. This method of the invention also produced a greater linear detection range than conventional ELISA method, making it more suitable for quantitation of low levels of ZAP-70 in clinical samples.

### Example V

### Assay Variability

The assay was validated for intra-assay variability and inter-assay variability (Table 3). To minimize the variation caused by cell lysis and sample preparation, multiple vials of Jurkat cell lysate with the same lot number were obtained from Millipore (Cat#12-303, Lot#DAM1641050) and tested by immunomagnetic fluorescence detection of ZAP-70. Two concentrations of Jurkat cell lysate, 6.25 μg/ml and 25 μg/ml, were repeatedly tested. A negative control of dilution buffer only was included in the experiment and subject to all assay procedures for measurement of the assay non-specific signals. A buffer control with Elution Buffer only was included in the final reading step for measurement of background noise caused by the Elution Buffer and Signalyte™-II instrument. Table 3 shows the resulting assay variability. Coefficient variations of intra-assay variability were 3.72%, 15.72% and 5.68% for the zero control, 6.25 μg/ml and 25 μg/ml of Jurkat cell lysate. Coefficient variations of inter-assay variability were 5.48%, 17.81% and 13.00% for the zero control, 6.25 μg/ml and 25 μg/ml of JCL.

TABLE 3
ASSAY VARIABILITY OF DETECTION OF ZAP-70 IN JURKAT CELL LYSATE

| | Intra-assay variability (n = 12) | | | | Inter-assay variability (n = 6) | | | |
|---|---|---|---|---|---|---|---|---|
| JCL (μg/ml) | Buffer | 0 | 6.25 | 25 | Buffer | 0 | 6.25 | 25 |
| | control | | | | control | | | |
| Average FI | 1595.50 | 2303.50 | 5501.25 | 18894.92 | 1643.93 | 2271.50 | 6586.04 | 18993.92 |
| Standard deviation | 22.42 | 85.80 | 864.65 | 1072.51 | 62.40 | 124.52 | 1173.04 | 2468.99 |
| Coefficient variation (%) | 1.41 | 3.72 | 15.72 | 5.68 | 3.80 | 5.48 | 17.81 | 13.00 |

JCL, Jurkat cell lysate;
FI, Fluorescence intensity;
Buffer control, a blank tube containing only the Elution Buffer which was used for evaluation of the background signals. To assess the intra-assay variability, twelve assays were performed on the same sample on the same day. To assess the inter-assay variability, the assays were performed on the same samples each day over 6 days.

### Example VI

### Detection of ZAP-70 in B Cells from Patients with CLL

Comparison of VeriZAP® with Flow Cytometry Analysis.

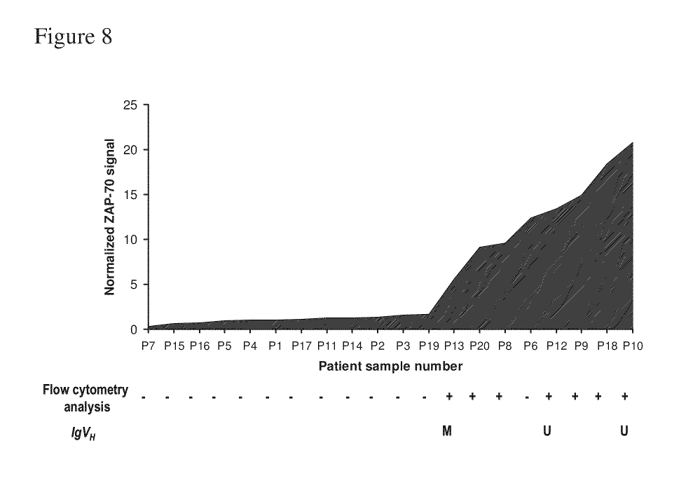

Twenty whole blood samples from patients with CLL were used for the comparative analysis (P1-P20). The ZAP-70 expression levels in these patients were determined by flow cytometry analysis as described (Degheidy H A et al., 2011a, Cytometry B Clin Cytom 80B:300-308; 2011b, Cytometry B Clin Cytom 80B:309-317). VeriZAP™ assay starts with preparation of B cell lysate from a fixed number of purified B cells from the CLL patients and followed by immunomagnetic fluorescence detection of ZAP-70. Purified T and B cell samples from a healthy control were used as positive and negative controls (PC and NC) for calculation of ZAP-70 ratio of each CLL cell sample. ZAP-70 expression was reported as the ratio of the average NFI of a CLL sample to the average NFI of the healthy control. ZAP-70 expression was defined as positive, if CLL/NC ratio ≧2.0 and as negative if the CLL/NC <2.0. The results of flow cytometry analysis and VeriZAP™ are summarized in FIG. 8. All 7 samples that had been determined ZAP-70-positive by flow cytometry were also determined as ZAP-70-positive by VeriZAP™ (100%). Further, 12 of 13 samples that had been determined as ZAP-70 negative by flow cytometry were also found negative by VeriZAP™. Only one sample of the 20 (P6) was discordant, measuring negative by flow cytometry, but positive by VeriZAP™, for an overall concordance of 95% between the two methods. We performed further analysis on the discrepant P6 sample to determine the ZAP-70-specific mRNA transcription level by quantitative RT-PCR. The results showed that the ZAP-70-mRNA transcription level in B cells from P6 was 7.84-fold higher than that in B cells from the healthy control, indicating that patient P6 had mRNA levels consistent with increased ZAP-70 expression. This demonstrated that the method of invention is more sensitive and specific than the conventional flow cytometry method. Overall, the flow cytometry and VeriZAP™ methods were highly correlated, confirming that VeriZAP™ is a valid alternative method for detection of ZAP-70 in CLL. In CLL B cells, elevated ZAP-70 expression appears to predict the need for therapy as effectively as IgVH mutation status. Although ZAP-70 expression is strongly correlated with IgVH mutation status, the combination of the 2 markers may provide greater prognostic value than either marker alone.

1. A method of diagnosing chronic lymphocytic leukemia (CLL) in a subject, comprising
preparing a ZAP-70 ratio M/N, wherein the ratio is prepared by quantitatively determining the amount of ZAP-70 M in a lysate of a predetermined number of B cells from a test subject, and dividing the determined amount M by a control amount N, wherein the control amount N is obtained by quantitatively determining the amount of ZAP-70 in a lysate of B cells of the same predetermined number from a control subject without CLL,
wherein when the ZAP-70 ratio M/N is greater than about 2.0, the test subject is diagnosed as having an aggressive form of CLL, and wherein when the ZAP-70 ratio M/N is less than about 2.0 the test subject is diagnosed as having an indolent form of CLL.

2. The method of claim 1, wherein the control amount N is obtained by quantitatively determining the amount of ZAP-70 in a lysate of B cells of the same predetermined number from a pool of control subjects without CLL.

3. The method of claim 1, wherein the amount of ZAP-70 in a lysate is determined by:
a) collecting a predetermined number of B cells,
b) lysing the B cells,
c) capturing ZAP-70 from the cell lysate of b), and
d) measuring the amount of ZAP-70 captured in c).

4. The method of claim 3, wherein the B cells are collected using a technique selected from the group consisting of immunomagnetic separation, Ficoll gradient followed by immunomagnetic separation, depletion of non-B cells (negative selection), and fluorescence-activated cell sorting (FACS).

5. The method of claim 3, wherein ZAP-70 is captured using one or more members selected from the group consisting of capture antibody-coated magnetic beads, aptamer-coated magnetic beads, ligand-coated magnetic beads, capture antibody-coated glass beads, aptamer-coated glass beads, ligand-coated glass beads, a capture antibody-containing ferrofluid, an aptamer-containing ferrofluid, a ligand-containing ferrofluid, a capture antibody-coated microfluidic chip, an aptamer-coated microfluidic chip and a ligand-coated microfluidic chip.

6. The method of claim 3, wherein the amount of ZAP-70 is measured by fluorescent sandwich assay or electrochemiluminescence.

7. The method of claim 6, wherein the fluorescent sandwich assay uses a detector antibody labeled with a detectable label selected from the group consisting of a fluorescent dye, a dye particle and quantum dots.

8. The method of claim 6, wherein a Signalyte®-II spectrofluorometer detection instrument, fluorescent plate reader or microfluidic chip is used to measure the amount of detectable label in the fluorescent sandwich assay.

9. The method of claim 5, wherein ZAP-70 is captured using capture antibody-coated magnetic beads and the capture antibody is selected from the group consisting of antibody 2F3.2, antibody 1E7.2, and SBZAP.

10. The method of claim 7, wherein the detector antibody is selected from the group consisting of antibody 2F3.2, antibody 1E7.2, and SBZAP.

11. The method of claim 7, wherein the fluorescence dye is selected from the group consisting of Texas Red, allophycocyanin (APC), Cy™-5, PE-Cy5.5, Dylight 649, DyLight 638/658, DyLight 654/673, DyLight 692/712, Alexa Fluor 590/617, Alexa Fluor 612/626, Alexa Fluor 632/647, Alexa Fluor 633/647, Alexa Fluor 650/665, and Alexa Fluor 663/690.

12. A method of diagnosing chronic lymphocytic leukemia (CLL) in a subject, comprising
(a) preparing a normalized ZAP-70 value X, wherein the normalized value is prepared by (i) quantitatively determining the amount of ZAP-70 M in a lysate of a predetermined number of B cells from a test subject, (ii) quantitatively determining the amount of a control marker P in the same lysate, and (iii) dividing the amount determined for ZAP-70 by the amount determined for the control marker to obtain the normalized ZAP-70 value where X=M/P,
(b) preparing a reference ZAP-70 value Y, wherein said reference value is prepared by (i) quantitatively determining the amount of ZAP-70 N in a lysate of B cells of the same predetermined number from a control subject without CLL, (ii) quantitatively determining the amount of the control marker R in the same lysate, and (iii) dividing the amount determined for ZAP-70 by the amount determined for the control marker to obtain the reference ZAP-70 value where Y=N/R, and
(c) obtaining a ZAP-70 ratio X/Y by dividing the normalized ZAP-70 value X by the reference ZAP-70 value Y,
wherein when the ZAP-70 ratio X/Y is greater than about 2.0, the test subject is diagnosed as having an aggressive form of CLL, and wherein when the ZAP-70 ratio X/Y is less than about 2.0 the test subject is diagnosed as having an indolent form of CLL.

13. The method of claim 12, wherein the lysate of B cells of the same predetermined number is from a pool of control subjects without CLL.

14. The method of claim 12, wherein the control marker is a protein selected from the group consisting of a tyrosine kinase protein, a human leukocyte differentiation antigen, and a transmembrane receptor proteins.

15. The method of claim 12, wherein the amounts of ZAP-70 and control marker in a lysate of a predetermined number of B cells are determined by:
a) collecting a predetermined number of B cells,
b) lysing the B cells,
c) capturing ZAP-70 and the control marker from the cell lysate of b), and
d) measuring the amount of ZAP-70 and the control marker captured in c).

16. The method of claim 15, wherein the B cells are collected using a technique selected from the group consisting of immunomagnetic separation, Ficoll gradient followed by immunomagnetic separation, depletion of non-B cells (negative selection), and fluorescence-activated cell sorting (FACS).

17. The method of claim 15, wherein ZAP-70 and the control marker are independently captured using one or more members selected from the group consisting of capture antibody-coated magnetic beads, aptamer-coated magnetic beads, ligand-coated magnetic beads, capture antibody-coated glass beads, aptamer-coated glass beads, ligand-coated glass beads, a capture antibody-containing ferrofluid, an aptamer-containing ferrofluid, a ligand-containing ferrofluid, a capture antibody-coated microfluidic chip, an aptamer-coated microfluidic chip and a ligand-coated microfluidic chip.

18. The method of claim 15, wherein the amount of ZAP-70 and the control marker are independently measured by fluorescent sandwich assay or electrochemiluminescence.

19. The method of claim 18, wherein the fluorescent sandwich assay uses a detector antibody labeled with a fluorescent material selected from the group consisting of a fluorescent dye, a dye particle and quantum dots.

20. The method of claim 18 or 19, wherein a Signalyte®-II spectrofluorometer detection instrument, fluorescent plate reader or microfluidic chip is used to measure the amount of fluorescence in the fluorescent sandwich assay.

21. The method of claim 17, wherein ZAP-70 is captured using capture antibody-coated magnetic beads and the capture antibody is selected from the group consisting of antibody 2F3.2, antibody 1E7.2, and SBZAP.

22. The method of claim 19, wherein the detector antibody is selected from the group consisting of antibody 2F3.2, antibody 1E7.2, and SBZAP.

23. The method of claim 19, wherein the fluorescence dye is selected from the group consisting of Texas Red, allophycocyanin (APC), Cy™-5, PE-Cy5.5, Dylight 649, DyLight 638/658, DyLight 654/673, DyLight 692/712, Alexa Fluor 590/617, Alexa Fluor 612/626, Alexa Fluor 632/647, Alexa Fluor 633/647, Alexa Fluor 650/665, and Alexa Fluor 663/690.

24. A method of diagnosing chronic lymphocytic leukemia (CLL) in a subject, comprising
a) preparing a ZAP-70 ratio M/N, wherein the ratio is prepared by quantitatively determining the amount of ZAP-70 M in a lysate of a predetermined number of B cells from a test subject by the following steps:
i) collecting a predetermined number of B cells from the test subject,
ii) lysing the B cells of i),
iii) capturing ZAP-70 from the B cell lysate of ii) using magnetic beads coated with a capture antibody, wherein the capture antibody has binding specificity for ZAP-70,
iv) forming an immunosandwich complex by adding a detector antibody to iii), wherein the detector antibody has binding specificity for ZAP-70 and wherein the detector antibody is labeled with a fluorescent dye,
v) collecting the magnetic beads from iv), and
vi) detecting and measuring fluorescent signal in v), wherein the amount of fluorescent signal corresponds to the amount of ZAP-70 M,
b) dividing the determined amount M by a control amount N, wherein the control amount N is obtained by quantitatively determining the amount of ZAP-70 in a lysate of B cells of the same predetermined number from a control subject without CLL by the following steps:
i) collecting B cells of the same predetermined number from a control subject or pool of control subjects,
ii) lysing the B cells of i),
iii) capturing ZAP-70 from the B cell lysate of ii) using magnetic beads coated with a capture antibody, wherein the capture antibody has binding specificity for ZAP-70,
iv) forming an immunosandwich complex by adding a detector antibody to iii), wherein the detector antibody has binding specificity for ZAP-70 and wherein the detector antibody is labeled with a fluorescent dye,
v) collecting the magnetic beads from iv), and
vi) detecting and measuring the amount of fluorescent signal in v), wherein the amount of fluorescent signal corresponds to the control amount N,
wherein when the ZAP-70 ratio M/N is greater than about 2.0, the test subject is diagnosed as having an aggressive form of CLL, and wherein when the ZAP-70 ratio M/N is less than about 2.0 the test subject is diagnosed as having an indolent form of CLL.

25. The method of claim 24, wherein the capture antibody is independently selected from the group consisting of antibody 2F3.2, antibody 1E7.2, and SBZAP.

26. The method of claim 24, wherein the detector antibody is independently selected from the group consisting of antibody 2F3.2, antibody 1E7.2, and SBZAP.

27. The method of claim 7, wherein a Signalyte®-II spectrofluorometer detection instrument, fluorescent plate reader or microfluidic chip is used to measure the amount of detectable label in the fluorescent sandwich assay.

28. The method of claim 19, wherein a Signalyte®-II spectrofluorometer detection instrument, fluorescent plate reader or microfluidic chip is used to measure the amount of fluorescence in the fluorescent sandwich assay.
